(19) United States
(12) Patent Application Publication
(10) Pub. No.: US 2010/0199996 A1
(43) Pub. Date: Aug. 12, 2010

(54) Anti-Slip Structure of Respirator

(76) Inventor: Kuo Tsung Fu, Hsin Kang Township (TW)

Correspondence Address: KAMRATH & ASSOCIATES P.A., 4825 OLSON MEMORIAL HIGHWAY, SUITE 245, GOLDEN VALLEY, MN, 55422, US

(21) Appl. No.: 12/366,737

(22) Filed: Feb. 6, 2009

(51) Int. Cl. A61M 16/06

(52) U.S. Cl. 128/206.21

(57) ABSTRACT

An anti-slip structure of a respirator is provided. The respirator has a mask body whose top side is provided with a duct-connecting portion and whose lower end is peripherally provided with a closely fitting pad. In addition, the mask body has an outer surface provided with anti-slip portions, formed as concave recesses or raised patterns, on two opposite lateral sides of the mask body for being gripped by fingers. By pinching at the anti-slip portions on two opposite lateral sides of the mask body with fingers, an operator can handle the respirator easily and prevent the duct-connecting portion or the closely fitting pad from being tainted by bacteria on the fingers. Thus, not only can those parts of the respirator that are in direct or indirect contact with a patient remain in an uncontaminated and safe state, but also the operator can adjust the position of the respirator facilely.

### BACKGROUND OF THE INVENTION

1. Technical Field

The present invention relates to a design to facilitate handling of a pollution-free respirator in a safe manner. More particularly, the present invention relates to an anti-slip structure of a respirator wherein the anti-slip structure comprises a mask body having two opposite lateral sides provided respectively with concave or raised anti-slip portions for being gripped by fingers.

2. Description of Related Art

Generally, medical respirators such as oxygen respirators, anesthesia respirators, and so on have slightly different shapes and configurations depending on the equipment they utilize, their purposes, or situations where they are used. Referring to FIG. 1 for a conventional respirator 1, the respirator 1 comprises a mask body 11 having a top side provided with a duct-connecting portion 12 connected with a gas duct. The mask body 11 has a lower end peripherally provided with an air cushion-type, closely fitting pad 13. In order to secure the respirator 1 in position to a patient's face, a strap ring 2 is typically mounted around a base of the duct-connecting portion 12 so that a resilient strap is hooked up to the strap ring 2 and then fastened around the patient's head. Thus, the respirator 1 is secured in place so as to cover the patient's mouth and nose.

However, the respirator 1 is usually contained in a pollution-free package before use. An operator trying to open the pollution-free package and take the respirator 1 out of the package has to grip the duct-connecting portion 12 or the closely fitting pad 13 with his/her fingers because the mask body 11 has a curved and smooth outer surface that is difficult to handle. Therefore, the operator's hand or the glove thereon, which might have been tainted by pollutants in the operating environment, tend to contaminate the duct-connecting portion 12 or the closely fitting pad 13 so that the patient wearing the respirator 1 is unnecessarily infected via direct contact with the closely fitting pad 13 or via inhaling through the gas duct connected with the duct-connecting portion 12. Hence, medical equipment is often a source of nosocomial infections.

### BRIEF SUMMARY OF THE INVENTION

As stated above, the conventional respirator may be contaminated by the operator's hand when being taken out from its package. To solve this problem, the present invention proposes a solution in which a respirator comprises a mask body having, in addition to a top side provided with a duct-connecting portion and a lower end provided with a closely fitting pad, two opposite lateral sides provided respectively with anti-slip portions for being gripped by fingers.

Also, the aforesaid anti-slip portions of the present invention are formed on a surface of the mask body as concave recesses or strip-like raised patterns.

Therefore, the anti-slip portions provided respectively on the two opposite lateral sides of the mask body of the respirator according to the present invention and configured for being pinched by an operator's fingers allow the operator to avoid contact with the duct-connecting portion or the closely fitting pad. Thus, the risk of the patient's being infected by a source of contamination through the operator's manual contact with the duct-connecting portion or the closely fitting pad is significantly reduced. Meanwhile, the operator is also allowed to adjust a position of the respirator with ease.

### BRIEF DESCRIPTION OF THE SEVERAL VIEWS OF THE DRAWINGS

The invention as well as a preferred mode of use, further objectives and advantages thereof will be best understood by referring to the following detailed description of illustrative embodiments in conjunction with the accompanying drawings, wherein:

FIG. 1 is a perspective view of a conventional respirator;

FIG. 2 is a perspective view of a respirator according to the present invention;

FIG. 3 is a partially sectional view of the respirator according to the present invention; and

FIG. 4 illustrates a second embodiment of anti-slip portions according to the present invention.

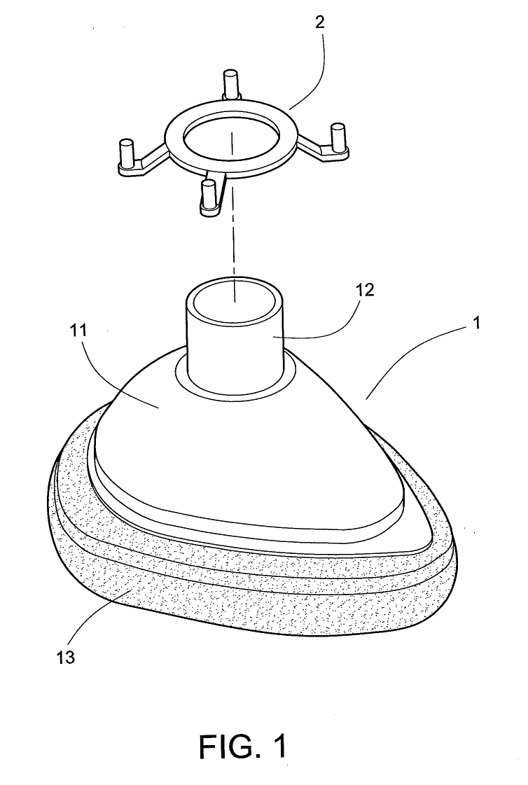

### DETAILED DESCRIPTION OF THE INVENTION

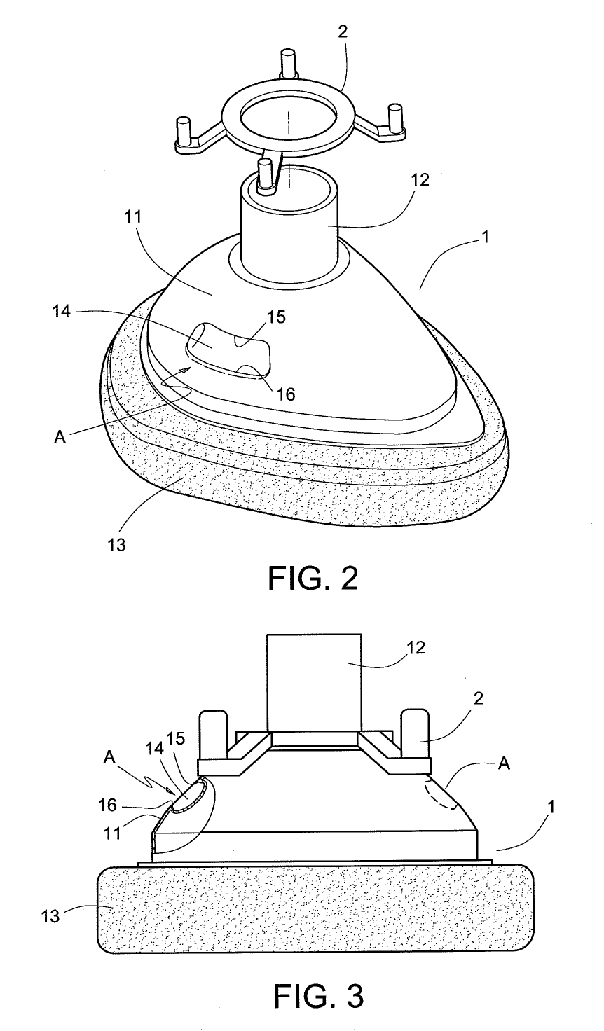

Referring to FIGS. 2 and 3, a respirator 1 according to the present invention comprises a mask body 11. The mask body 11 has a top side provided with a duct-connecting portion 12 connected with a gas duct. The mask body 11 also has a lower end peripherally provided with a closely fitting pad 13. A strap ring 2 to be hooked up with a resilient strap is mounted around a base of the duct-connecting portion 12. In addition, anti-slip portions A are provided on a surface of the mask body 11 on two lateral sides of the mask body 11. The anti-slip portions A are two concave recesses 14 corresponding in position to each other, each having an upper edge and a lower edge provided respectively with flanges 15 and 16 for being hooked by fingertips.

Therefore, the recesses 14 of the anti-slip portions A on the two opposite lateral sides of the mask body 11 of the respirator 1 allow an operator to insert his/her fingers thereinto so as to pinch the respirator 1 firmly. In this way, the operator's fingers come into contact only with the mask body 11 but not the duct-connecting portion 12 or the closely fitting pad 13 of the respirator 1. Consequently, the closely fitting pad 13 which is in direct contact with a patient or the duct-connecting portion 12 connected with the gas duct is kept at a pollution-free state, thereby significantly reducing the risk of nosocomial infections where the patient is infected through the respirator 1 by the operator's potentially polluted hands. Besides, if the respirator 1 is not properly positioned on the patient's face, the operator is allowed to grip the anti-slip portions A so as to adjust the position of the respirator 1.

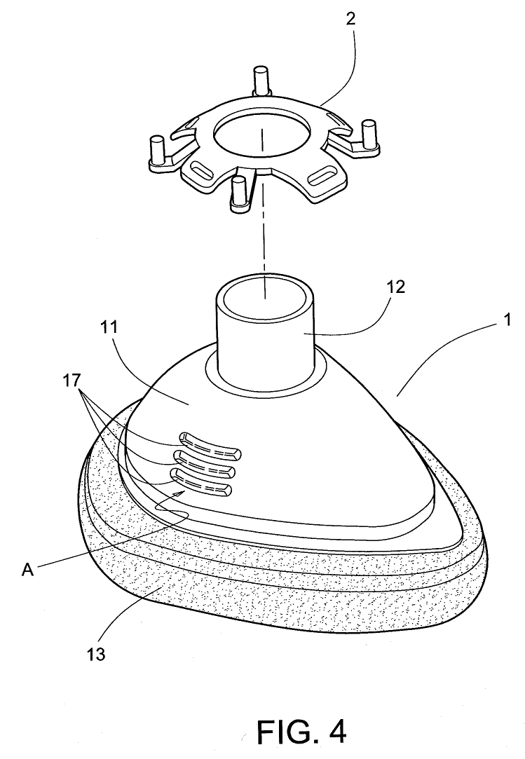

Referring to FIG. 4, the anti-slip portions A on the two opposite lateral sides of the mask body 11 are in the form of a plurality of raised patterns 17 configured to be gripped by the operator's fingers and serve an anti-slip purpose.

1. An anti-slip structure of a respirator, comprising a mask body having a top side provided with a duct-connecting portion, a lower end peripherally provided with a closely fitting pad, and an outer surface provided with anti-slip portions formed as concave recesses on two opposite lateral sides of the mask body for being gripped by fingers.

2. The anti-slip structure of the respirator as claimed in claim 1, wherein the recesses are provided with flanges at least at upper edges of the recesses.

3. An anti-slip structure of a respirator, comprising a mask body having a top side provided with a duct-connecting portion, a lower end peripherally provided with a closely fitting pad, and an outer surface provided with anti-slip portions formed as raised patterns on two opposite lateral sides of the mask body for being gripped by fingers.
